(19) United States
(12) Patent Application Publication
(10) Pub. No.: US 2020/0345657 A1
(43) Pub. Date: Nov. 5, 2020

(54) CANNABINOID STOCK FOR FORMULATION PRODUCTS

(71) Applicant: BetterLife Pharma Inc., Vancouver (CA)

(72) Inventors: Leonid Lurya, Rehovot (IL); Joseph Borovsky, Wilmington, NC (US)

(21) Appl. No.: 16/865,318

(22) Filed: May 2, 2020

Related U.S. Application Data
(60) Provisional application No. 62/841,848, filed on May 2, 2019.
(60) Provisional application No. 63/005,423, filed on Apr. 5, 2020.

(51) Int. Cl. A61K 31/05; A61K 9/06; A61K 36/886; A61K 31/125; A61K 31/045; A61K 31/355; A61K 36/324; A61K 36/28; A61K 8/9794; A61K 8/9789; A61K 8/73; A61K 8/9728; A61K 8/362

(52) U.S. Cl. CPC A61K 31/05; A61K 9/06; A61K 36/886; A61K 31/125; A61K 31/045; A61K 31/355; A61K 8/362; A61K 36/28; A61K 8/9794; A61K 8/9789; A61K 8/735; A61K 8/9728; A61K 36/324

(57) ABSTRACT

Transdermal cannabinoid formulations including a versatile cannabinoid stock provided as a variety of cosmetic delivery systems providing enhanced cannabinoid absorption, and more controlled release into vascular and/or lymphatic systems for greater efficacy and a desirable subject experience. The cannabinoid stock is a substantially homogeneous concentrated stable cannabinoid emulsion characterized as a cannabinoid load capacity carrier that comprises emulsified particles of stabilized cannabinoid/lipid.

### TECHNICAL FIELD

The invention relates to a cannabinoid stock adaptable for making cosmetic delivery systems for a variety of applications.

### BACKGROUND

Medical cannabis-based medicaments show much as yet unfulfilled promise in alleviating many medical conditions. The cannabinoid compounds are varied, and different extracts have different physiological, clinical and receptor binding effects. Most medical cannabis is smoked or taken orally and in many cases, is found not to be as effective as desired.

Topical formulations have been developed, but are inadequate. The currently available formulations suffer, however, from a number of drawbacks, including lack of suitability of the carrier for its intended use. Most of these known formulations suffer from an inability to carry a large amount of the cannabinoid active agent and to ensure a controlled and prolonged release thereof at the desired site. This inability is particularly undesirable, since usually any biologically active agent must remain at the desired site for a prolonged period in order to be effective.

The discussion of the background herein is included to explain the context of the inventions described herein. This is not to be taken as an admission that any of the material referred to was published, known, or part of the common general knowledge as of the priority date of any of the claims.

### SUMMARY

It is desired to provide cannabinoid delivery systems to achieve enhanced cannabinoid absorption, efficacy and subject experience.

It is also desired to provide a cannabinoid base as a versatile vehicle for making a variety of cosmetic delivery systems providing enhanced cannabinoid absorption, and more controlled release into vascular and/or lymphatic systems for greater efficacy and a desirable subject experience.

In an aspect, is a versatile cannabinoid stock. The cannabinoid stock is a substantially homogeneous concentrated stable cannabinoid emulsion characterized as a cannabinoid load capacity carrier that comprises emulsified particles of stabilized cannabinoid/lipid. The emulsion is substantially thermodynamically and/or kinetically stable such that the size of the stabilized cannabinoid/lipid particles does not substantially change over time.

The cannabinoid stock is suitable for direct transdermal application or for transdermal application as incorporated as the base into a cosmetic delivery system.

In aspects, the cannabinoid stock is for making a variety of cosmetic delivery systems for different applications. The cosmetic delivery systems are for topical use/application for transdermal delivery of the cannabinoid.

The cannabinoid stock and cosmetic delivery systems have a pH compatible with pH of the biomembrane to which they will be delivered (skin or mucosa).

The cannabinoid stock is versatile as it can be made containing a range of concentration of the cannabinoid and admixed in a desired amount/ratio with a cosmetic formulation comprising ingredients (components) to make a specific type (format) of cosmetic delivery system such as but not limited to a cream, lotion, gel, ointment, liquid, balm, oil and solid to provide a product for specific application with a desired amount/concentration of cannabinoid. The cannabinoid/lipid particles remain stable both in the stock and as incorporated in the cosmetic formulation and the selection of the format of cosmetic formulation may vary the absorption of the cosmetic formulation through dermis and/or mucosal surface and thus the release time of the cannabinoid from the cannabinoid/lipid particles.

The cannabinoid stock stably entrains a desired amount of cannabinoid by comparison to conventional delivery compositions, and the smooth uniform characteristic of the stock emulsion provides versatility to produce a variety of cosmetic delivery systems with more consistent cannabinoid distribution and concentration, for more effective transdermal delivery of the cannabinoid. The cannabinoid stock of the invention as incorporated within a desired cosmetic formulation is advantageous for improved bioavailability of the cannabinoid whether in a cream, lotion, gel, ointment, liquid, balm, oil or solid formulation.

Cosmetic formulations comprising the cannabinoid stock of the invention are for topical administration for transdermal delivery of the stabilized cannabinoid/lipid particles therein to reach subdermal layers for releasing cannabinoid into the systemic circulation. The method of making the cannabinoid stock incorporates a judicious selection of lipid and stabilizer combined with cannabinoid and process steps to make emulsified cannabinoid/lipid particles optimally sized for absorption through dermal layers and through mucosal layers. The stability of the cannabinoid/lipid particles is maintained in the emulsion and in the cosmetic delivery system. Thereafter as applied to the skin, the bioavailable size of the cannabinoid/lipid particles remain intact and thus can reach into the hypo dermis of the skin where cannabinoid depots are formed—are passively accumulated. As the lipid is slowly metabolized cannabinoid is released into vasculature and lymphatic system. Additionally, lymphatic delivery is beneficial as it bypasses the first-pass metabolism in the liver thus increasing bioavailability of the cannabinoid.

The cannabinoid stock and cosmetic delivery systems made with the stock enable more effective dosaging with concomitant enhancement of cannabinoid bioavailability and reduced variability of the absorption/bioavailability levels between different cosmetic formulation products and between subjects using their preferred format of cosmetic formulation.

As an additive to a cosmetic formulation, the cannabinoid stock can be stored until ready for use to manufacture a cosmetic delivery system. Given that different formats of cosmetic delivery systems comprise a variety of different ingredients/chemicals, some of the additives may interact with each other. Whilst the ingredients/chemicals may not necessarily chemically react with one another, some of them may not optimally mix well together to stay shelf stable for a long period of time. It is possible that over time an undesirable generation of haze and/or sediment and/or physical property of the cosmetic format may occur.

The cannabinoid stock that comprises a stable substantially homogeneous concentrated cannabinoid emulsion can be used as an additive combined a cosmetic base to make a cosmetic formulation (e.g. cream, lotion, gel, ointment, liquid, balm, oil, or solid) in amounts to provide a desired concentration of the cannabinoid in the cosmetic format without negatively affecting the organoleptic properties of the cosmetic formulation or the bioavailability of the cannabinoid. In this manner the resultant product has more desirable properties, most notably, provides a substantially effective transdermal source of cannabinoid at desired concentrations regardless of the cosmetic format.

The cannabinoid emulsion comprises stabilized cannabinoid/lipid particles in an average diameter size in the range of up to about 10 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 41 nm to about 45 nm, about 42.5 nm to about 47.5 nm, about 45 nm to about 50 nm, about 47.5 nm to about 52.5 nm, about 50 nm to about 55 nm, about 52.5 nm to about 57.5 nm, about 55 nm to about 60 nm, about 57.5 nm to about 62.5 nm, about 60 nm to about 65 nm, about 62.5 nm to about 67.5 nm, about 65 nm to about 70 nm, about 67.5 nm to about 72.5 nm, about 70 nm to about 75 nm, about 72.5 nm to about 77.5 nm, about 75 nm to about 80 nm, about 77.5 nm to about 82.5 nm, about 80 nm to about 85 nm, about 82.5 nm to about 87.5 nm, about 85 nm to about 90 nm, about 87.5 nm to about 92.5 nm, about 90 nm to about 95 nm, about 92.5 nm to about 97.5 nm, about 95 nm to about 99 nm, about 10 nm to about 310 nm, about 50 nm to about 300 nm and combinations thereof.

According to an aspect of the invention is a cannabinoid stock. The cannabinoid stock is a cannabinoid load capacity carrier.

According to an aspect of the invention is a cannabinoid stock that is a cannabinoid load capacity carrier comprising emulsified particles of stabilized cannabinoid/lipid particles, wherein the lipid and the cannabinoid are present in a ratio from about 5:1 to about 1:5.

The cannabinoid stock advantageously has a continuous phase comprising or consisting of water.

According to a further aspect of the invention is a cannabinoid stock emulsion comprising stabilized cannabinoid/lipid particles, wherein the lipid and the cannabinoid are present in a ratio from about 3:1 to about 1:3.

According to an aspect of the invention are stable cannabinoid/lipid particles, the particles comprising cannabinoid, lipid, stabilizer and anti-oxidant, wherein the lipid and the cannabinoid are present in a ratio from about 5:1 to about 1:5 or 3:1 to about 1:3.

In any aspect the cannabinoid/lipid particles comprise micelles, mixed micelles and micelle aggregates. In aspects, the micelles are substantially uniform in size. In aspects, the micelles are about 10 nm to about 300 nm inclusive of any size in between. In aspects the cannabinoid/lipid particles comprise liquid particles of about 50 nm to about 300 nm.

In aspects the cannabinoid is CBD and/or THC. In further aspects the cannabinoid is CBD.

In aspects, the cannabinoid stock comprises up to about 10% by weight of cannabinoid. In further aspects up to about 9% by weight, up to about 8% by weight, up to about 7% by weight, up to about 6% by weight, up to about 5% by weight, up to about 4% by weight, up to about 3% by weight, up to about 2.5% by weight, up to about 2% by weight, up to about 1.5% by weight, up to about 1% by weight, and up to about 0.5% by weight cannabinoid, in aspects CBD.

In aspects the lipid is a phospholipid and can be selected from soy lecithin, rapeseed lecithin, corn or sunflower lecithins, egg lecithin, Epicorn™ 200, Phosal™ 50 PG, dioleyl phospatidylcholine (DOPC), oleyl palmytoyl phosphatidylcholine (POPC), and the corresponding serines, ethanol amines, glycerolphosphatidylcholine. In aspects, the lipid is phosphatidylcholine. In aspect the lipid is provided in a ratio with the cannabinoid of about 5:1 to 1:5, in aspects about 4:1 to 1:4, in aspects about 3:1 to 1:3.

In aspects the stabilizer is a surfactant. In aspects the surfactant is water miscible. In aspects the surfactant is propylene glycol. In aspects the cannabinoid stock lipid phase comprises up to about 50% by weight of stabilizer. In aspects the cannabinoid stock comprises up to about 15% by weight stabilizer.

In aspects, the anti-oxidant is a water soluble anti-oxidant. In aspects, the water soluble anti-oxidant is vitamin E (for example a derivative of vitamin E, d-α-tocopherol such as for example vitamin E TPGS (d-α-tocopheryl polyethylene glycol 1000 succinate). In aspects the cannabinoid stock comprises up to about 1.0% by weight of anti-oxidant. In aspects, the lipid phase of the stock comprises up to about 0.5% by weight anti-oxidant or up to about 0.2% by weight vitamin E/TPGS.

In aspects of the invention, the cannabinoid stock comprises an oil phase that comprises or consists of CBD, phosphatidylcholine, propylene glycol and vitamin E/TPGS.

In aspects of the invention, the cannabinoid stock comprises or consists of a lipid phase of up to about 10% by weight CBD, about 45% by weight phosphatidylcholine, about 45% by weight propylene glycol and about 0.2% by weight vitamin E/TPGS.

In aspects of the invention, the cannabinoid stock comprises:

a lipid phase of about 10% by weight CBD, about 45% by weight phosphatidylcholine, about 45% by weight propylene glycol and about 0.2% by weight vitamin E/TPGS; and

a water phase.

In aspects, the cannabinoid stock comprises a substantially homogeneous emulsion of stabilized CBD/lipid particles. In aspects, the emulsion comprises or consists of water as the continuous phase.

In aspects, the cannabinoid stock comprises a substantially homogeneous concentrated emulsion of stabilized CBD/lipid particles. In aspects, the emulsion comprises or consists of water as the continuous phase.

According to an aspect of the invention is a method of making a cannabinoid stock comprising:
preparing a lipid phase by intimately admixing at an elevated temperature, a stabilizer, a lipid, one or more cannabinoids and an anti-oxidant;
high shear mixing the lipid phase with a water phase at an elevated temperature; and
subjecting the mixture to cycles of high pressure homogenization until a smooth and substantially uniform emulsion is formed.

In aspects, the uniform emulsion is cooled to about room temperature for storage and/or use.

In aspects, the intimate admixing can be done for up to about 30 minutes.

In aspects, the high shear mixing is done for about 1 minute up to about 60 minutes inclusive of any integer of time in between.

In aspects the lipid phase is prepared at a temperature range of about 45° C. to about 60° C., in aspects, about 50° C. to about 55° C., in aspects about 55° C.

In aspects the shear mixing is done at temperatures of about 45° C. to about 60° C., in aspects, about 50° C. to about 55° C., in aspects about 45° C. or 55° C.

In aspects, the high pressure homogenization is conducted at pressures of up to about 20,000 psi. In aspects about 3,000 psi to about 20,000 psi. In aspects at least about 3,000 psi, at least about 4,000 psi, at least about 5,000 psi, at least about 6,000 psi, at least about 7,000 psi, at least about 8,000 psi, at least about 9,000 psi, about 10,000 to about 20,000 psi, in aspects about 10,000 to about 15,000 psi or in aspects any pressure contained within the approximate ranges.

In aspects, the lipid phase is admixed with water in ratios of about 5:1 to 1:5.

In a further embodiment is a method for making a cannabinoid stock comprising:

preparing a lipid phase by dissolving phosphatidylcholine in heated propylene glycol, thereafter adding cannabinoid and water soluble antioxidant and mixing;

mixing at high shear the lipid phase with a heated water phase; and

applying high pressure homogenization for a plurality of cycles to form a smooth and substantially homogeneous cannabinoid emulsion.

The cannabinoid stock is stable at a variety of temperatures, humidity levels and light conditions for a prolonged period of time. The cannabinoid stock can be stored for up to about 3 months at temperatures of about 19° C. to about 25° C.

The present invention specifically meets many needs by providing a cannabinoid stock that is versatile in that it can be admixed to make a cosmetic delivery system that can be of a variety of different types of cosmetic formulations such as a cream, a lotion, a liquid, a solid, a gel and the like without substantially affecting the stability of the cannabinoid/lipid particles. The cannabinoid stock carries a stable load of cannabinoid in an emulsified format that can readily blend with different types of cosmetic formulations. The cosmetic delivery system can be made with any amount of cannabinoid stock and blended with cosmetic components/ingredients to provide a cannabinoid cosmetic product comprising up to about 10%, 9%, 8%, 7%, 6%, 5%, 4.5%, 4%, 3.5%, 3%, 2.5%, 2%, 1.5%, 1% and 0.5% by weight cannabinoid. The cosmetic delivery system may be selected from the group consisting of cream, oil, balm, lotion, gel, ointment, liquid and solid formulation.

Unexpectedly, the cannabinoid stock according to the invention is advantageously physico-chemically stable and makes it possible, by virtue of the various components and physical characteristics, to make a large number of cosmetic formulations with different textures that can effectively provide transdermal cannabinoid.

Further, the cannabinoid stock by virtue of its stability properties can be advantageously stored without substantial degradation. In addition, the cannabinoid stock can be used, for example mixed with selected cosmetic components/ingredients in order to form an immediate cosmetic delivery system. Advantageously, the cannabinoid stock makes it possible to obtain fresh cosmetic delivery systems, making it possible to substantially reduce/prevent the possible deterioration of the cannabinoid active(s).

In aspects, the cosmetic delivery system is a cosmetic type formulation comprising components/ingredients to make a cream, balm, lotion, gel, oil, ointment, liquid (for dispensing as a liquid, drops, spray, aerosol or foam) and a solid (e.g. solid stick or suppository). By admixing with the cannabinoid stock in a variety of ratios a desired amount of cannabinoid active is achieved with desired organoleptic properties and consistency for dermal and mucosal applications. It is within the scope of the invention that the cosmetic type formulation also comprise an amount of cannabinoid, in aspects up to 5% by weight of its components.

The cosmetic delivery systems of the invention are suitable for continued use and multiple applications with minimal to no dermal or mucosal irritation or damage. They may comprise any desired amount of one or more cannabinoid depending on the amount present in the cannabinoid stock and the ratio admixed with the cannabinoid stock.

In aspects is a system for making a cosmetic delivery system, the system comprising a first component comprising a cannabinoid stock and a second component comprising a cosmetic formulation, wherein the first component is admixed with the second component to make the cosmetic delivery system. The cosmetic formulation comprises ingredients to make a cream, lotion, gel, balm, liquid, ointment, oil or solid stick. In aspects, the cosmetic formulation can also comprise cannabinoid such as CBD. In this aspect, both the cannabinoid stock and the cosmetic formulation comprise CBD prior to admixing.

Creams, lotions, gels, balms, liquids, ointments, oils or solid stick can be made in any variety of volume product amounts such as for example 50 mls, 100 mls, 150 mls, 200 mls, 250 mls and up to about 500 mls or more. Each volume product can comprise a desired amount of cannabinoid such as CBD. For example a 100 mls gel lubricant composition can be formulated to contain up to about 150 mg CBD, or up to about 200 mg CBD or more. The amounts of CBD in the product will vary according to the amount of CBD in the cannabinoid stock and the amount if any of CBD in the volume product and the volume used.

Kits comprising the cannabinoid stock and packaging and/or instructions for use are within the scope of the invention.

The cosmetic formulation comprising the cannabinoid stock is for topical use to the skin or a mucosal surface to provide transdermal cannabinoid useful for alleviating a condition, for improving undesired symptoms of a condition, for providing a general feeling of wellness/calm/pleasure, for reducing anxiety, and/or increasing mental buoyancy without inducing abnormal behavior or other adverse effects.

According to an aspect of the invention is a method for treating pain in a subject, the method comprising topical application of a cosmetic delivery system comprising the cannabinoid stock of the invention. In aspects for alleviating acute or chronic pain. The acute or chronic pain can be a side-effect of a treatment or a condition. The cosmetic delivery system is used to alleviate pain or discomfort in a subject by being applied to the skin of the subject thereby causing the cannabinoid in the formulation to pass into and/or through the skin of the subject. In aspects, the cosmetic formulation can be applied to a patch form for the treatment of the pain and discomfort associated for example, but not limited to menstrual cramps, water retention (e.g., “bloating”) and/or muscular pain (e.g., muscular back pain).

In non-limiting aspects is a the cosmetic delivery system incorporating the cannabinoid stock for topical application as a skin care product for application to the dermis and/or a mucosal surface.

In a non-limiting aspect the cosmetic delivery system comprising a cannabinoid stock is a gel for topical application to the dermis and/or mucosa for alleviating pain.

In a non-limiting aspect the cosmetic delivery system comprising a cannabinoid stock is a cream or lotion for topical application to the dermis and/or mucosa for alleviating pain or for providing a feeling of well being.

In a non-limiting aspect cosmetic delivery system comprising a cannabinoid stock is a lubricant (for example, water-based, silicone-based, hydroxyethylcellulose-based or organic) for topical application to the dermis and/or mucosa.

According to an aspect of the invention is a lubricant comprising or consisting of a substantially homogeneous concentrated emulsion of stabilized CBD/lipid particles.

According to a further aspect is a cannabinoid stock containing lubricant composition for lubricating mucous membranes, the cannabinoid stock comprising or consisting of a lipid phase of up to about 10% by weight CBD, about 45% by weight phosphatidylcholine, about 45% by weight propylene glycol and about 0.2% by weight vitamin E/TPGS.

In aspects, is a lubricant comprising a cannabinoid stock comprising or consisting of a lipid phase of up to about 10% by weight CBD, about 45% by weight phosphatidylcholine, about 45% by weight propylene glycol and about 0.2% by weight vitamin E/TPGS.

According to a further aspect is a method for providing lubrication and pain relief and/or a feeling of well-being to a mucosal surface, the method comprising applying a lubricant comprising a cannabinoid stock comprising or consisting of a lipid phase of up to about 10% by weight CBD, about 45% by weight phosphatidylcholine, about 45% by weight propylene glycol and about 0.2% by weight vitamin E/TPGS, to the mucosal surface.

According to a further embodiment of the invention, is a formulation for application to the skin, the formulation comprising: (a) a biologically active agent, and (b) a lipid, wherein the biologically active agent is selected from the group consisting of Dronabinol (2), Nabiximols, Nabilone, THC, CBD, Cannabidiol, Levonantradol, Ajulemic acid, (CT3), ECP002A, Natural Δ9-THC, Cannabichromenes, Cannabichromene (CBC), Cannabichromenic acid (CBCA), Cannabichromevarin (CBCV), Cannabichromevarinic acid (CBCVA), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoins, Cannabielsoic acid B (CBEA-B), Cannabielsoin (CBE), Cannabielsoin acid A (CBEA-A), Cannabigerols, Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA, Cannabinols and cannabinodiols, Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriols, 10-Ethoxy-9-hydroxy-delta-6α-tetrahydrocannabinol,8,9-Dihydroxy-delta-6α-tetrahydrocannabinol, Cannabitriol (CBT), Cannabitriolvarin (CBTV), Delta-8-tetrahydrocannabinols, Delta-8-tetrahydrocannabinol (Δ⁸-THC), Delta-8-tetrahydrocannabinolic acid (Δ⁸-THCA), Delta-9-tetrahydrocannabinols, Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA) 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triOH-THC), 3,4,5,6-Tetrahydro-7-hydroxy-alpha-alpha-2-trimethyl-9-n-propyl-2,6-methano-2H-1-benzoxocin-5-methanol, OH-iso-HHCV and combinations thereof.

In further aspects of the formulation, the lipid and the biologically active agent are present in a ratio from about 5:1 to about 1:5 or from about 3:1 to about 1:3, with the ranges inclusive of any integer and fractions thereof found within the ranges. Further, the ranges, integer and fractions thereof can be approximate.

In further aspects, the formulation comprises a stabilizer comprising at least one surfactant selected from the group consisting of non-ionic, anionic, cationic, and amphiphilic surfactant. The non-ionic surfactant may be selected from the group consisting of polyethylene glycol (PEG), a PEG derivative and a glycerol derivative. The PEG derivative may be selected from the group consisting of alpha-hydro-omega-hydroxypoly-(oxy-1,2-ethanediyl), polyethylene glycol mono[4-(1,1,3,3-tetramethylbutyl) phenyl]ether, propylene glycol, O-3-Amino-3-deoxy-D-glucopyranosyl-(14)-O-[2,6,diamino-2,3,6-trideoxy-D-ribo-hexopyransol-(16)]-2-deoxy-L-streptamine, alpha-hydro-omega-hydroxpoly(oxyethylene)poly(oxypropylene)poly(oxyethylene) block copolymers, polyethylene glycol fatty alcohol ethers, sorbitan fatty acid esters, poloxamer, and polyethylene glycol esters of fatty acids. The glycerol derivative may be selected from the group consisting of alpha-hydro-omega-hydroxypoly(oxy-1,2-ethanediyl) and polyalkylglyceride.

The anionic surfactant may be selected from the group consisting of carboxylate, alkyl sulfonate, aryl sulfonate and phosphate. The cationic surfactant may be selected from the group consisting of alkyl pyridinium salt and tetraalkylammonium salt. The amphiphilic surfactant may be selected from the group consisting of alkyl betaine derivative, cocoamphodiacetate derivative, trimyristin, trilaurin, tripalmitin, tristearin, and phosphatidylglycerol.

The formulation may further comprise at least one lipid additive selected from the group consisting of triglyceride, alkyl ester, cholesterol, octadecenoic acid 1,2,3-propanetriyl ester, edible oil, tetradecanoic acid 1-methylethyl ester, and methyl ester beta-Cholest-5-en-3-ol.

The formulation may further comprise at least one additive selected from the group consisting of flavor, aroma modifier, sweetener, color, and antioxidant.

Further embodiments of the present invention provide the aforementioned formulation as a colloidal dispersion comprising micelles, mixed micelles, and micellar aggregates comprising the cannabinoid. The micelle can have an average diameter of from about 10 nm to about 300 nm (inclusive of any integer therein).

Further embodiments of the present invention provide the aforementioned formulation where the lipid is in the form of a dispersion containing cannabinoid liquid particles of size in the range of from about 50 nm to about 300 nm (inclusive of any integer therein).

According to an aspect of the invention is a method for making a subdermal cannabinoid depot in a subject, the method comprising topically applying a cosmetic delivery system selected from a cream, gel, liquid, solid, balm, foam and paste to an area of skin on the subject, wherein the cosmetic delivery system comprises a cannabinoid stock comprising up to about 5% by weight cannabinoid as emulsified particles of stabilized cannabinoid/lipid that penetrate subdermally to form the cannabinoid depot.

In aspects, the cannabinoid depot is metabolized to release cannabinoid into the vascular and/or lymph system of the subject.

In aspects, the cream, gel, liquid, solid, balm, foam and paste may also comprise up to about 5% by weight cannabinoid.

In further embodiments in the aforementioned formulation the biologically active agent may have systemic activity being suitable for treatment of at least one condition selected from the group consisting of inflammation, irritation, dryness, and microbial infection, nausea and vomiting due to chemotherapy, appetite stimulation in HIV/AIDS, chronic pain, spasticity due to multiple sclerosis or paraplegia, depression, anxiety disorder, sleep disorder, psychosis, glaucoma, Tourette syndrome, neuropathic pain (central, peripheral), cancer pain, diabetic peripheral neuropathy, fibromyalgia, refractory pain due to MS or other neurological conditions, rheumatoid arthritis, non-cancer pain (nociceptive and neuropathic), central musculoskeletal problems, and chemotherapy-induced pain.

Aspects of the invention include but are not limited to:

1. A cannabinoid stock comprising a stabilized cannabinoid/lipid particle emulsion, wherein the lipid and the cannabinoid are present in a ratio from about 5:1 to about 1:5.
2. The cannabinoid stock disclosed above in 1, wherein the ratio is from about 3:1 to about 1:3.
3. The cannabinoid stock disclosed above in 1 or 2, wherein water forms the continuous phase.
4. The cannabinoid stock of any one disclosed above in 1-3, wherein the lipid is a phospholipid and can be selected from soy lecithin, rapeseed lecithin, corn or sunflower lecithins, egg lecithin, Epicorn™200, Phosal™ 50 PG, dioleyl phospatidylcholine (DOPC), oleyl palmytoyl phosphatidylcholine (POPC), and the corresponding serines, ethanol amines, glycerolphosphatidylcholine.
5. The cannabinoid stock disclosed above in 4, wherein the lipid is phosphatidylcholine.
6. The cannabinoid stock disclosed above in 5, wherein the phosphatidylcholine comprises up to about 50% by weight of the cannabinoid stock.
7. The cannabinoid stock of any one disclosed above in 1-6, comprising a stabilizer that stabilizes the cannabinoid/lipid particles.
8. The cannabinoid stock disclosed above in 7, wherein the stabilizer is water miscible.
9. The cannabinoid stock disclosed above in 8, wherein the stabilizer is a surfactant.
10. The cannabinoid stock disclosed above in 9, wherein the surfactant is propylene glycol.
11. The cannabinoid stock of any one disclosed above in 7-11, comprising up to about 50% by weight of stabilizer.
12. The cannabinoid stock of any one disclosed above in 1-11, comprising a water soluble anti-oxidant.
13. The cannabinoid stock disclosed above in 12, wherein the water soluble anti-oxidant is vitamin E (alpha-tocopherol).
14. The cannabinoid stock disclosed above in 13, wherein the cannabinoid stock comprises up to about 5% by weight of the anti-oxidant.
15. The cannabinoid stock of any one disclosed above in 1-14, comprising an oil phase that comprises or consists of CBD, phosphatidylcholine, propylene glycol and vitamin E.
16. The cannabinoid stock of any one disclosed above in 1-15, wherein the cannabinoid is CBD and/or THC.
17. The cannabinoid stock disclosed above in 16, wherein the cannabinoid is CBD.
17a. The cannabinoid stock of any one disclosed above in 1-17 wherein the particles are about 10 nm to about 300 nm or about 50 nm to about 300 nm.
18. A transdermal formulation comprising a cannabinoid stock according to any one of 1-17a disclosed above and a cosmetic base comprising a cosmetic formulation selected from the group consisting of cream, lotion, gel, ointment, liquid, solid stick and foam.
18a. The transdermal formulation disclosed above in 18, wherein the cream, lotion, gel, ointment, liquid, solid stick and foam comprises CBD.
19. The transdermal formulation disclosed above in 18 and 18a, wherein the liquid is further formulated for use as an aerosol spray.
20. The transdermal formulation disclosed above in 18 and 18a, wherein the liquid is further formulated for use as a foam.
21. A cannabinoid stock comprising a substantially homogeneous emulsion of CBD/lipid particles, propylene glycol and vitamin E.
22. A method of making a cannabinoid stock comprising:
preparing a lipid phase by intimately admixing at an elevated temperature, a stabilizer, a lipid, one or more cannabinoids and an anti-oxidant;
high shear mixing the lipid phase with a water phase at an elevated temperature; and
subjecting the mixture to cycles of high pressure homogenization until a smooth and substantially uniform emulsion is formed.
   23. A method of making a cannabinoid stock comprising:

preparing a lipid phase by dissolving phosphatidylcholine in heated propylene glycol, thereafter adding cannabinoid and water soluble antioxidant and mixing;

mixing at high shear the lipid phase with a heated water phase; and

applying high pressure homogenization for a plurality of cycles to form a smooth and substantially homogeneous cannabinoid emulsion.

24. The method disclosed above in 22 or 23, wherein up to about 50% by weight cannabinoid stock is admixed with up to about 50% by weight of a cosmetic base comprising ingredients to make a cosmetic formulation selected from the group consisting of cream, oil, balm, lotion, gel, ointment, liquid and solid formulation.
25. A cannabinoid emulsion comprising:

(a) a first lipid phase comprising stabilized cannabinoid lipid particles; and

(b) a second water phase,

wherein the lipid phase particles are substantially homogeneously suspended in the first lipid phase, and

wherein the lipid and the cannabinoid are present in a ratio from about 5:1 to about 1:5.

26. The cannabinoid emulsion disclosed above in 25, wherein the cannabinoid is selected from the group consisting of natural or synthetic cannabinoid, tetrahydrocannabinols (THC), Δ⁹-THC, ⁹-THC Propyl Analogue (THC-V); Cannabidiol (CBD); Cannabidiol Propyl Analogue (CBD-V); Cannabinol (CBN), Cannabichromene (CBC); cannabinodiol (CBDL); cannabicyclol (CBL); Cannabichromene Propyl Analogue (CBC-V); cannabielsoin (CBE); cannabitriol (CBT), Cannabigerol (CBG), pharmaceutically acceptable salts of these cannabinoids, cannabinoid prodrugs, cannabinoid agonists, synthetic analogs thereof and combinations thereof.
27. The cannabinoid emulsion disclosed above in 26, wherein the cannabinoid is CBD.
28. The cannabinoid emulsion disclosed above in 27, wherein CBD is present in an amount of up to about 50% by weight of the emulsion.
29. The cannabinoid emulsion of any one of 25-28 disclosed above, wherein the stabilized cannabinoid lipid particles comprise an anti-oxidant and/or stabilizer.
30. The cannabinoid emulsion of any one of 25-29 disclosed above, wherein at least 30% of said cannabinoid is entrapped within the particles.
31. The cannabinoid emulsion of any one of 25-30 disclosed above, wherein the cannabinoid is entrapped within and between phospholipid bilayers of the cannabinoid particles.
32. The cannabinoid emulsion of any one of 25-31 disclosed above formulated as a cream, lotion, liquid, gel, foam, drops, suppository, ointment, spray or patch.
33. The cannabinoid emulsion disclosed above in 32, wherein said formulation comprises up to 5% by weight CBD, up to 4% CBD, up to 3% by weight CBD, up to 2% by weight CBD or up to 1% by weight CBD.
33a. The cannabinoid emulsion disclosed above in 33, wherein the cream, lotion, liquid, gel, foam, drops, suppository, ointment, spray or patch initially comprises CBD.
34. A method for the treatment of pain in a subject comprising transdermal administration of the transdermal formulation of any one of 18-20 disclosed above or the cannabinoid emulsion of any one of 25-33a disclosed above to an area of pain on skin or a mucosal surface.
35. The method disclosed above in 34, wherein said formulation or emulsion may be administered repeatedly to said skin.
36. The method disclosed above in 34 or 35, wherein said formulation or emulsion is vigorously massaged into the skin to raise the skin temperature such that the composition may penetrate pores in the epidermis of the skin.
37. A method for the treatment of pain in a subject comprising topically administering the transdermal formulation of any one of 18 to 20 disclosed above or the cannabinoid emulsion of any one of 25 to 33 disclosed above to mucosa of the mouth, nose, vagina or rectum.
38. The transdermal formulation of any one of 18 to 20 disclosed above formulated as a cream comprising up to 5% by weight CBD and a base cream formulation.
39. The formulation of 38 disclosed above, wherein said CBD is provided in an amount of up to about 1% by weight, up to about 2% by weight, up to about 3% by weight or up to about 4% by weight.
40. The formulation of 32 disclosed above, wherein said cream formulation comprises at least two ingredients selected from the group consisting of water, ceteraryl octanoate, glycerin, shea butter, sweet almond oil, palm oil, jojoba oil, aloe barbaensis, mans sal, potassium sorbate, sclerotium gum, xanthum gum, tocopheryl acetate, Camellia sinensis leaf extract, corral powder and any combination thereof.
41. A cannabinoid cosmetic formulation for topical administration to the skin or mucosa, the formulation comprising the cannabinoid stock of any one of 1 to 17a disclosed above, wherein said cannabinoid is provided in a therapeutically effective amount for alleviating pain.
42. The cannabinoid formulation of 41 disclosed above, wherein said cannabinoid is CBD.
43. The cannabinoid formulation of 41 or 42 disclosed above, wherein said composition penetrates the epidermal layer of compromised skin and/or penetrates the mucosa.
44. The cannabinoid formulation of any one of 41 to 43 disclosed above, comprising up to about 10% by weight CBD.
45. A formulation for application to the skin, the formulation comprising: (a) a biologically active agent and (b) a lipid wherein said biologically active agent is selected from the group comprising Dronabinol (2), Nabiximols, Nabilone, THC, CBD, Cannabidiol, Levonantradol Ajulemic acid, (CT3), ECP002A, Natural Δ9-THC, Cannabichromenes, Cannabichromene (CBC), Cannabichromenic acid (CBCA), Cannabichromevarin (CBCV), Cannabichromevarinic acid (CBCVA), Cannabidiol Cannabidiol (CBD), Cannabidiol monomethylether (CBDM), Cannabidiolic acid (CBDA), Cannabidiorcol (CBD-C1), Cannabidivarin (CBDA), Cannabidivarinic acid (CBDVA), Cannabielsoins, Cannabielsoic acid B (CBEA-B), Cannabielsoin (CBE), Cannabielsoin acid A (CBEA-A), Cannabigerols, Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA, Cannabinols and cannabinodiols, Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool, (CBN-C1), Cannabivarin (CBV), Cannabitriols, 10-Ethoxy-9-hydroxy-delta-6a-tetrahydrocannabinol,8,9-Dihydroxy-delta-6a-tetrahydrocannabinol, Cannabitriol (CBT), Cannabitriolvarin (CBTV), Delta-8-tetrahydrocannabinols, Delta-8-tetrahydrocannabinol (Δ⁸-THC), Delta-8-tetrahydrocannabinolic acid (A-THCA), Delta-9-tetrahydrocannabinols, Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannahivarinic acid (THCVA) 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triOH-THC), 3,4,5,6-Tetrahydro-7-hydroxy-alpha-alpha-2-trimethyl-9-n-propyl-2,6-methano-2H-1-benzoxocin-5-methanol, or OH-iso-HHCV.
46. The formulation of 45 disclosed above, wherein said lipid in (b) and said biologically active agent in (a) are present in a ratio from about 5:1 to about 1:5.
47. The formulation of 45 or 46 disclosed above, further comprising a stabilizer, said stabilizer having at least one surfactant selected from the group consisting of non-ionic, anionic, cationic, and ampiphilic.
48. The formulation of 45, 46 or 47 disclosed above, wherein said lipid is in a colloidal dispersion of a form selected from the group consisting of micelles, mixed micelles, and micellar aggregates, said lipid having a particle size of from about 10 to about 300 nm.
49. The formulation of 45, 46 or 47 disclosed above, wherein said lipid is in the form of a dispersion having liquid particles of size in the range of from about 50 to 300 nm.
50. A lubricant cosmetic formulation comprising a cannabinoid stock comprising a substantially homogeneous cannabinoid emulsion of about 10% by weight CBD combined with a mixture of ingredients selected from the group consisting of Natrosol 250HHR, Vanzan NF, Aloe Vera Gel, Zemea (Propanediol), Hemp Extract, Sodium Hyaluronate, Quinoa Seed Extract GL, Linseed Extract GL, Green Tea Extract GL, shitake Mushroom Extract, Oat Kernel Extract GL, Citric Acid 20% solution, Geogard Ultra, Potassium Sorbate, NaOH 20% solution, and Sodium Benzoate.
51. A pain gel formulation comprising a cannabinoid stock comprising or consisting of a substantially homogeneous cannabinoid emulsion of about 10% by weight CBD combined with a mixture of ingredients selected from the group consisting of Lecigel, Glycerin, Allantoin, Sodium Hyaluronate, Camphor, Menthol, Vitamin E Acetate (Tocopherol acetate), Arnica Extract GL, Boswellia Extract GL, Aloe Veral Gel 10× (Ale Barbadensis Leaf Juice) and Euxyl PE 9010.
52. A lubricant comprising or consisting of a substantially homogeneous concentrated emulsion of stabilized CBD/lipid particles.
52a. The lubricant of 52 disclosed above, provided in a volume of about 100 ml to about 500 mls.
53. The lubricant of 52a disclosed above, provided in a volume of about 100 mls and comprising about 50 mg to about 200 mg CBD.
53. A cannabinoid stock containing lubricant composition for lubricating mucous membranes, the cannabinoid stock comprising or consisting of a lipid phase of up to about 10% by weight CBD, about 45% by weight phosphatidylcholine, about 45% by weight propylene glycol and about 0.2% by weight vitamin E/TPGS.
54. A method for providing lubrication and pain relief and/or a feeling of well-being to a mucosal surface, the method comprising applying a lubricant comprising a cannabinoid stock comprising or consisting of a lipid phase of up to about 10% by weight CBD, about 45% by weight phosphatidylcholine, about 45% by weight propylene glycol and about 0.2% by weight vitamin E/TPGS, to the mucosal surface.
55. A method for providing cannabinoid depots in the hypodermis of skin, the method comprising applying a cream or lotion comprising a cannabinoid stock formulation of cannabinoid/lipid particles directly to an area of skin, wherein the cannabinoid/lipid particles remain intact and reach into the hypo dermis of the skin forming the cannabinoid depots.
56. The method of 55 disclosed above, wherein the lipid is slowly metabolized to release the cannabinoid into vasculature and lymphatic system.
57. The method of any one of 54 to 56 disclosed above, wherein the cannabinoid stock comprises a lipid phase of up to about 10% by weight CBD, about 45% by weight phosphatidylcholine, about 45% by weight propylene glycol and about 0.2% by weight vitamin E/TPGS.
58. A cannabinoid stock containing cream composition for providing a depot of cannabinoid in subdermal layers of skin, the cream composition comprising a cannabinoid stock comprising or consisting of a lipid phase of up to about 10% by weight CBD, about 45% by weight phosphatidylcholine, about 45% by weight propylene glycol and about 0.2% by weight vitamin E/TPGS.
59. The cannabinoid stock containing cream composition of 58 disclosed above, wherein the lipid is slowly metabolized to release the cannabinoid into vasculature and lymphatic system.
60. A kit comprising the transdermal formulation of any one of 1 to 17a disclosed above, and instructions for use and/or packaging.

61. The kit of 60 disclosed above, wherein the transdermal formulation is formatted in amounts of about 100 mls, about 150 mls, about 200 mls, about 250 mls, about 300 mls, about 350 mls, about 400 ml, about 450 ml and about 500 ml.

62. The kit of 61 disclosed above, wherein each formatted amount may comprise up to about 500 mg, up to about 400 mg, up to about 300 mg, up to about 200 mg, up to about 150 mg, or up to about 100 mg cannabinoid.
63. A system for making a cosmetic formulation comprising cannabinoid, the system comprising:

(a) a transdermal formulation of any one of 1 to 17 disclosed above; and

(b) a cream, gel, lotion, ointment, liquid, solid stick or foam formulation,

wherein (a) is admixed with (b) in a desired volume ratio.

### BRIEF DESCRIPTION OF THE DRAWINGS

The above and other aspects, advantages, and features of this disclosure will become more apparent by describing in further detail exemplary embodiments thereof with reference to the accompanying drawings in which:

FIG. 1 shows release of cannabinoid from the sub dermal layer showing zero order release;

FIG. 2 shows release of cannabinoid from the sub dermal layer showing first order release;

FIG. 3 shows release of cannabinoid from the sub dermal layer showing loading and sustained release;

FIG. 4 shows release of cannabinoid from the sub dermal layer showing delayed and sustained release;

FIG. 5 shows release of cannabinoid from the sub dermal layer showing delayed and pulsatile release;

FIG. 6 shows release of cannabinoid from the sub dermal layer showing pulsatile release; and

FIG. 7 shows release of cannabinoid from the sub derma layer showing ascending release.

### DETAILED DESCRIPTION

As used herein, the terms “invention” or “present invention” are non-limiting terms and not intended to refer to any single aspect of the particular invention but encompass all possible aspects as described in the specification and the claims.

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control.

Unless defined otherwise, all technical and scientific terms used herein have the same meaning as is commonly understood by one of skill in the art to which the subject matter herein belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the following definitions are supplied in order to facilitate the understanding of the present invention.

It will be understood that any component defined herein as being included may be explicitly excluded from the claimed invention by way of proviso or negative limitation. In addition, all ranges given herein include the end of the ranges and also any intermediate range points, whether explicitly stated or not.

As used herein, the articles “a” and “an” preceding an element or component are intended to be non-restrictive regarding the number of instances (i.e. occurrences) of the element or component. Therefore, “a” or “an” should be read to include one or at least one, and the singular word form of the element or component also includes the plural unless the number is obviously meant to be singular.

It will be further understood that the terms “comprises” and/or “comprising,” or “includes”, “including” and/or “having” and their inflections and conjugates denote when used in this specification, specify the presence of stated features, regions, integers, steps, operations, elements, and/or components, but do not preclude the presence or addition of one or more other features, regions, integers, steps, operations, elements, components, and/or groups thereof.

It will be understood that any component defined herein as being included may be explicitly excluded from the claimed invention by way of proviso or negative limitation.

As used herein, the term “about” refers to variation in the numerical quantity. In one aspect, the term “about” means within 10% of the reported numerical value. In another aspect, the term “about” means within 5% of the reported numerical value. Yet, in another aspect, the term “about” means within 10, 9, 8, 7, 6, 5, 4, 3, 2, or 1% of the reported numerical value.

“About,” is equivalent to “approximately,” or “substantially” as used herein and inclusive of the stated value and means within an acceptable range of deviation for the particular value as determined by one of ordinary skill in the art, considering the measurement in question and the error associated with measurement of the particular quantity (i.e., the limitations of the measurement system). For example, “about,” “approximately,” or “substantially” can mean within one or more standard deviations, or within +30%, 20%, 10%, 5% of the stated value.

Should a range of values be recited, it is merely for convenience or brevity and includes all the possible sub-ranges as well as individual numerical values within and about the boundary of that range. Any numeric value, unless otherwise specified, includes also practical close values and integral values do not exclude fractional values. Sub-range values and practically close values should be considered as specifically disclosed values.

As will also be understood by one skilled in the art, all language such as “up to”, “at least”, “greater than”, “less than”, “more than”, “or more”, and the like, include the number recited and such terms refer to ranges that can be subsequently broken down into subranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

As used herein the term ‘may’ denotes an option or an effect which is either or not included and/or used and/or implemented and/or occurs, yet the option constitutes at least a part of some embodiments of the invention or consequence thereof, without limiting the scope of the invention.

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary.

As used herein, expressions such as “at least one of,” when preceding a list of elements, modify the entire list of elements and do not modify the individual elements of the list.

The term “transdermal patch” as used herein means a skin patch to be applied to the mammals skin containing the pharmaceutical composition. The technology for constructing transdermal patches is well known in the pharmaceutical art. The terms “backing layer” and “reservoir” as used herein are components of the transdermal patch. Suitable materials and designs are well known in the transdermal drug delivery art. See for example D. Hsien, “Multiple Lamination for Transdermal Patches,” Controlled Release Systems Fabrication Technology, Vol. 1, pp. 167-188. 1988.

As used herein “transdermal” describes absorption through the skin or mucosal membranes for systemic distribution. As placed on the skin, the cannabinoid stock or transdermal cosmetic format is capable of delivering cannabinoid through the stratum corneum layer of the epidermis and through the dermis into the microvasculature. As placed on a mucous membrane that lines several passages and cavities of the body with openings exposed to the external environment, the cannabinoid stock or transdermal cosmetic format comprising the cannabinoid stock is capable of delivering cannabinoid through the mucous membrane into the microvasculature.

As used herein, the phrase “transdermal delivery” means administration of the cannabinoid stock or cosmetic formulation comprising the cannabinoid stock topically to the skin or mucosal surface wherein the active ingredient, the cannabinoid, will be percutaneously delivered in a therapeutically effective amount.

“Combination or combining” for the purposes of this invention means any method of putting two or more materials together. Such methods include, but are not limited to, mixing, blending, commingling, concocting, homogenizing, incorporating, intermingling, fusing, joining, shuffling, stirring, coalescing, integrating, confounding, joining, uniting, or the like.

The term “pharmaceutically acceptable” means that the compound or combination of compounds is compatible with the remaining ingredients of the formulation for pharmaceutical use, and that it is generally safe for administering to humans according to established governmental standards.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

An “effective amount” refers to an amount effective to treat to which this phrase refers, this can be a disease, disorder, and/or condition, or to bring about a recited effect. Determination of a therapeutically effective amount is well within the capacity of persons skilled in the art. The term “effective amount” is intended to include an amount of a compound described herein, or an amount of a combination of compounds described herein for providing the recited effect to a subject. Thus, an “effective amount” generally means an amount that provides the desired effect.

As used herein “cannabinoid” is a class of chemical compounds that act on cannabinoid receptors on cells that repress neurotransmitter release in the brain, the cannabinoid receptors including the endocannabinoids, phytocannabinoids, synthetic cannabinoids, and cannabidiol, or combinations thereof.

Cannabinoids as used herein refers to any cannabinoid (natural or synthetic) and include phytocannabinoids and most of these fall into the subclasses such as cannabigerol, cannabichromene, cannabidiol, cannabinol (including tetrahydrocannabinol, e.g., Δ⁹-THC, Δ⁸-THC). Other cannabinoids include cannabicyclol, cannabielsoin, cannabinoldiol, and cannabitriol. Cannabinoids useful for the present invention include cannabinols. In one embodiment, the invention includes tetrahydrocannabinols, including tetrahydrocannabinol (THC), dronobinol, cannabinol (CBN) and (−)-trans-cannabidiol (CBD). Cannabinoids described herein are inclusive of their pharmaceutically acceptable salts.

Cannabinoids for use in the present invention in an aspect are selected from CBN, CBDA, CBD, THC, THCA, and mixtures thereof. Mixtures of CBD and THC can be, for example, 1:1 w/w or any other mixture. Various ratios of the above-described cannabinoids can be used for the cannabinoid stock and transdermal formulation described herein. The ratios can be adjusted based on pharmacological effects required. Ratios of CBD:THC are for example, 1:1, 0.1:1, 0.2:1, 0.3:1, 0.4:1, 0.5:1, 0.6:1, 0.7:1, 0.8:1, 0.9:1, 1:1, 1:1.2, 1:1.5, 1:1.3, 1:1.5, 1:1.7, 1:2, 1:3, 1:4, 1:5, 1:6, 1:7, 1:8 or 1:10 (all ratios given are w/w). In aspects, the cannabinoid stock comprises CBD and/or THC. In aspects, the cannabinoid stock comprises up to 50% by weight CBD and/or TI-IC.

“Alleviate” as used herein, is meant to include complete elimination as well as any clinically or quantitatively measurable reduction in the subject's symptoms and/or discomfort.

By pain as used herein is meant both acute and chronic. For example acute pain usually comes on suddenly and is caused by something specific. Acute pain usually does not last longer than six months. It goes away when there is no longer an underlying cause for the pain. Causes of acute pain include: surgery, broken bones, dental work, burns, cuts, strains, sprains, pain due to intercourse, menstruation and the like. Chronic pain is pain that is ongoing and usually lasts longer than six months. This type of pain can continue even after the injury or illness that caused it has healed or gone away. Pain signals remain active in the nervous system for weeks, months, or years. Some people suffer chronic pain even when there is no past injury or apparent body damage. Chronic pain is linked to conditions including but not limited to: headache, arthritis, cancer, nerve pain, scarring/scar tissue, back pain, fibromyalgia, bursitis, carpal tunnel syndrome, gout, tissue scarring and other muscular and joint aches and pains.

In aspects the cannabinoid stock can be applied to a patch (to form a cannabis transdermal delivery structure) that is constructed to have a backing layer selected from the group consisting of a patch, strip, bandage or covering, for example, the backing layer comprising the composition of the invention and optional other skin permeation enhancer(s) or other components. One of skill in the art would recognize that the composition described herein can be incorporated into a variety of patch formats such as for example but not limited to those disclosed in U.S. Pat. Nos. 6,113,940, 6,328,992 and 9,375,417 each of which are incorporated herein by reference in their entirety.

A general non-limiting overview of the invention and practising the invention is presented below. The overview outlines exemplary practice of embodiments/aspects of the invention, providing a constructive basis for variant and/or alternative and/or divergent aspects/embodiments, some of which are subsequently described.

### Transdermal Formulations of Cannabinoid-Based Medicaments

Drug delivery through the skin to achieve a systemic effect of a drug is commonly known as transdermal drug delivery and differs from traditional topical drug delivery. Transdermal drug delivery systems (TDDS) are dosage forms involves drug transport to viable epidermal and or dermal tissues of the skin for local therapeutic effect while a very major fraction of drug is transported into the systemic blood circulation. The adhesive of the transdermal drug delivery system is critical to the safety, efficacy and quality of the product. Topical administration of therapeutic agents offers many advantages over conventional oral and invasive methods of drug delivery. Several important advantages of transdermal drug delivery are limitation of hepatic first pass metabolism, enhancement of therapeutic efficiency and maintenance of steady plasma level of the drug. (International Journal of Pharmaceutical Sciences Review and Research, J. Ashok Kumar et al.).

Novel formulations are provided herein for carrying cannabis-based medicaments and cosmetic delivery systems containing cannabinoids and related compounds through the epidermis, through the dermis (containing at least lymph vessels) to the sub dermal lipid layer (i.e. the hypo dermis layer) that contains subcutaneous fat, such as the white adipose tissue (WDA) and blood vessels.

The transdermal delivery is achieved by topical application to any area of skin surface which carries the cannabinoid(s) through the epidermis to the dermal layer and then through to the sub dermal lipid layer where functional cannabinoid depots are formed. These functional cannabinoid depots comprise cannabinoid particles allowing precise controlled release of the cannabinoids into the vascular and/or lymphatic system of the subject. Administration of cannabinoids with multiple release times can be achieved by the carrier formulation components.

Transdermal delivery is also achieved by topical application to a mucosal surface (such as that lining the ears, inside the nose, inside the mouth, lip, vagina, the urethral opening and the anus) which carries the cannabinoid(s) through to the submucosa for cannabinoid delivery to the blood vessels. Administration of cannabinoids with multiple release times can be achieved by the cosmetic formulation components.

The invention relates to embodiments of a cannabinoid stock that is characterized as a cannabinoid load capacity carrier. The stock comprises emulsified particles of stabilized cannabinoid/lipid sized for enhanced absorption. The particles comprising desired ratios of lipid to cannabinoid. In an aspect of an embodiment, the cannabinoid stock is used to make a cosmetic delivery system for delivering the cannabinoid by topical application meaning the provision of a local effect, where the composition is applied directly where its action is desired. The term topical may be defined as application to a localized area of the body or to the surface of a body part, without necessarily involving a targeted effect of the substance, resulting in a systemic effect. Examples of topical administration/use includes, for example, transdermal and transmucosal delivery (e.g., by intravaginal administration, rectal, or intranasal). In aspects, there are also localized benefits from topical administration. For example, topically administered cannabinoids may find use in alleviating pain and other conditions originating near the surface of the skin. Transdermal includes application to any skin portion of the body.

Methods and systems for making the cannabinoid stock and the cosmetic delivery systems are within the scope of the invention.

The cannabinoid stock is generally made by combining a lipid phase containing the cannabinoid with a water phase which is then subjected to cycles of high pressure homogenization until the formation of a stable substantially homogeneous concentrated cannabinoid emulsion is formed in which the cannabinoid is entrapped within stable lipid particles of the emulsion resulting in the cannabinoid being less subject to degradation, hydrolysis and oxidation. The cannabinoid stock is suitable to use as an additive to formulate a cosmetic delivery system for topical use providing a release profile.

In one non-limiting aspect, the lipid phase is made by adding about 10% to about 30% by wgt of substantially pure CBD to a heated mixture of stabilizer and fatty acid (at a temperature of about 45° C. to about 55° C.) until well blended and then adding a water soluble antioxidant. This lipid phase is added to heated water (temperature of about 45° C. to about 50° C.) and subjected to about 1 to 6 cycles of high pressure homogenization at pressures of about 3,000 psi to about 20,000 psi. Each cycle being up to about 5 minutes. The final emulsion is stable and presents as smooth and uniform in consistency comprising cannabinoid particles that are liquid particles of a size of about 50 nm to about 300 nm, or micelles of a size of about 10 nm to about 300 nm.

The cannabinoid stock, as an additive, is admixed in a desired amount/ratio with a cosmetic formulation comprising components/ingredients that make a specific type of cosmetic delivery system such as a cream, lotion, gel, ointment, liquid, balm, oil and solid to provide an end product for a specific use with a desired amount/concentration of cannabinoid.

The cosmetic delivery system according to the present invention passively delivers cannabinoid and can release the cannabinoid for an extended time period by having long-term adhesion to the skin and by having a significantly improved skin penetration rate in comparison with other conventional formats. The judicious selection of lipid and cannabinoid and method to make the lipid/cannabinoid particle in the stabilized emulsion provides for increased absorption and thus better bioavailability of the cannabinoid. Without being bound by theory, the cosmetic delivery system comprising the cannabinoid stock penetrates the skin and the cannabinoid/lipid particles can penetrate into subdermal layers to form a depot within subcutaneous fat stores. This depot metabolizes over time releasing the cannabinoid into lymphatic and vasculature systems.

For transdermal delivery of the cannabinoid stock of the invention an amount of the cannabinoid stock is admixed with a desired cosmetic type formulation to provide a desired texture of a cosmetic delivery system comprising a lotion, cream, balm, gel, ointment, liquid, and solid. Contacting with the subject's skin is effective for at least one of the provided cannabinoids to penetrate into the skin and enter the bloodstream. The cannabinoid stock and cosmetic delivery systems incorporating the stock allow for significant transdermal delivery across skin and compromised skin.

A number of methods known in the art can be used to assess delivery across the skin. In one method, delivery may be assessed by measurement of the remaining cannabinoid in the composition after use. After the composition was present on the skin of a patient for at least 12 hours, for example, at least 0.1% of the cannabinoid can be delivered across the skin, at least 0.5% of the cannabinoid can be delivered across the skin, at least 1% of the cannabinoid can be delivered across the skin, at least 2% of the cannabinoid can be delivered across the skin, at least 3% of the cannabinoid can be delivered across the skin, at least 4% of the cannabinoid can be delivered across the skin, at least 5% of the cannabinoid can be delivered across the skin, at least 6% of the cannabinoid can be delivered across the skin, at least 7% of the cannabinoid can be delivered across the skin, at least 8% of the cannabinoid can be delivered across the skin, at least 9% of the cannabinoid can be delivered across the skin, at least 10% of the cannabinoid can be delivered across the skin, at least 11% of the cannabinoid can be delivered across the skin, at least 12% of the cannabinoid can be delivered across the skin, at least 14% of the cannabinoid can be delivered across the skin, at least 16% of the cannabinoid can be delivered across the skin, at least 18% of the cannabinoid can be delivered across the skin, at least 20% of the cannabinoid can be delivered across the skin, at least 25% of the cannabinoid can be delivered across the skin, at least 30% of the cannabinoid can be delivered across the skin, at least 35% of the cannabinoid can be delivered across the skin, at least 40% of the cannabinoid can be delivered across the skin, at least 45% of the cannabinoid can be delivered across the skin, at least 50% of the cannabinoid can be delivered across the skin, at least 55% of the cannabinoid can be delivered across the skin, at least 60% of the cannabinoid can be delivered across the skin, at least 65% of the cannabinoid can be delivered across the skin, at least 70% of the cannabinoid can be delivered across the skin, at least 75% of the cannabinoid can be delivered across the skin, at least 80% of the cannabinoid can be delivered across the skin, at least 85% of the cannabinoid can be delivered across the skin, at least 90% of the cannabinoid, at least 90% of the cannabinoid can be delivered across the skin, and at least 95% of the cannabinoid can be delivered across the skin.

### Cosmetic Delivery Systems

The cosmetic delivery systems described herein will typically include the cannabinoid stock and one or more other ingredients to provide different cosmetic formulations.

For topical administration, it will generally be desirable to administer the cosmetic delivery system directly to the skin/mucosal surface or can be applied from absorbent pads, used to impregnate bandages and other dressings, or sprayed onto a desired area using a pump-type or aerosol sprayer.

For topical application the cosmetic delivery system may comprise generally ingredients inclusive but not limited to lipid thickeners (e.g. Cetyl Alcohol, Stearyl Alcohol, Carnauba Wax, and Stearic acid), naturally derived thickeners (cellulose derivatives such as hydroxyethylcellulose, guar gum, xanthan gum and gelatin), mineral thickeners (e.g. Silica, Bentonite, and Magnesium Aluminum Silicate), synthetic thickeners (e.g. carbomer thickeners), alcohols, absorption promotors, fragrances, natural ingredients (e.g. aloe vera, cocoa butter, and coconut oil), scents (peppermint, cinnamon, menthol, jasmine), camphor, shea butter, gelling agents, emollients, synthetic preservatives (e.g. organohalogens, aldehydes, glycol ethers, parabens), natural preservatives (benzoic acid, sorbic acid, salicyclic acid and alcohol), synthetic antioxidants (e.g. butylated hydroxytoluene (BHT) and butylated hydroxyanidole (BHA)), natural antioxidants (e.g. tocopherol (Vitamin E), ascorbic acid (Vitamin C), polyphenols, and flavonoids).

More specifically, cosmetic formulation ingredients may include a “carrier” that is physiologically compatible with the skin or mucosal tissue of a human or animal to which it is topically administered. Typically the carrier is substantially inactive, with the exception of its intrinsic surfactant properties which may aid in the production of a solution or suspension of the active ingredients. In some embodiments, the carriers can be liquid or gel-based materials for use in liquid or gel formulations. Suitable carrier materials include any carrier or vehicle commonly used as a base for solutions, dispersions, emulsions, gels, creams, ointment, lotions, pastes, or foams, for topical administration. Examples include emulsifying agents, inert carriers including hydrocarbon bases, emulsifying bases, non-toxic solvents or water-soluble bases.

Suitable liquid or gel-based carriers are may include water, physiological salt solutions, alcohols (e.g., methanol, ethanol, propanol, or butanol), glycerol, glycols (e.g., ethylene glycol, propylene glycol, or ethoxy diglycol), polyethylene glycol (e.g., MW 400 to 20,000), water-alcohol/glycol blends, and the like. Suitable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (together or in various combinations), and detergents (e.g., polysorbates (Tweens) such as polysorbate 20, 40, 60, or 80; polyoxyl stearate; or sodium lauryl sulfate). One or more carrier materials can be mixed with water to form a lotion, gel, cream, semi-solid composition, or the like. Other suitable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate. glycerin, mineral oil, synthetic spermaceti, cetyl alcohol, or combinations thereof, may be used. Preservatives may also be included in the carrier, such as one or more of butylparaben, methylparaben, propylparaben, benzyl alcohol, and ethylene diamine tetraacetate salts. The composition of the carrier can be varied so long as it does not interfere significantly with the stability of the emulsified cannabinoid/lipid particles.

In one embodiment, the carrier can be a PLO gel (pluronic lecithin organogel). PLO gel contains isopropyl palmitate (a non-oleaginous emollient), soy lecithin (mixture of phospholipids), water, and Pluronic F127.

The cosmetic formulation components/ingredients may comprise gelling agents and thickening agents to increase the viscosity of the cosmetic delivery system. Examples of gelling agents and thickening agents, include, but are not limited to, fatty acids, fatty acid salts and esters, fatty alcohols, synthetic polymers, modified celluloses, xanthan gum, or combinations thereof. Examples of suitable synthetic polymers include polyethylene glycol (PEG), polyvinylpyrrolidone (PVP), polyvinyl alcohol (PVA), various Pluronics (poloxamers), or carbomers (e.g., Carbomer 940 or Carbomer 934). Examples of suitable modified celluloses include methylcellulose, carboxymethylcellulose (CMC), hydroxyethylcellulose (HEC), hydroxymethyl cellulose (HMC), hydroxypropyl cellulose (HPC), hydroxypropyl-methylcellulose (HPMC), or other cellulose-based gelling agents.

A variety of gelling agents is commercially available and can be obtained in many suitable molecular weights and ranges. For example, the molecular weights of the gelling agent can be about 1 kDa to about 1,000 kDa, about 10 kDa to about 1,000 kDa, about 100 kDa to about 1,000 kDa, or about 50 kDa to about 500 kDa.

Examples of thickening agents include lanolin, hard paraffin, liquid paraffin, white petrolatum, soft yellow paraffin or soft white paraffin, white beeswax, yellow beeswax, propolis (propoleum), cetostearyl alcohol, cetyl alcohol, dimethicones, emulsifying waxes, microcrystalline wax, oleyl alcohol and stearyl alcohol.

One or more gelling agents or thickening agents may be included in a single cosmetic delivery system and further used to form spreadable gels, pastes, ointments and the like, for application directly to the skin of the user.

Solutions and dispersions of cosmetic delivery systems of the invention can be prepared in water, optionally mixed with a nontoxic surfactant. Dispersions can be prepared in glycerol, liquid polyethylene glycols, triacetin, or in a pharmaceutically acceptable oil or mixtures thereof. Under ordinary conditions of storage and use, preparations may contain a preservative to prevent the growth of microorganisms. The liquid carrier or vehicle can be a solvent or liquid dispersion medium comprising, for example, water, ethanol, a polyol (for example, glycerol, propylene glycol, liquid polyethylene glycols, and the like), vegetable oils, emu oil, nontoxic glyceryl esters, and suitable mixtures thereof. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the cosmetic delivery system can be brought about by agents delaying absorption, for example, aluminum monostearate and/or gelatin.

Solutions can be prepared by incorporating the cannabinoid stock in a desired amount in the appropriate solvent or oil with various other ingredients described herein, as desired, followed by optional filter sterilization.

Gels are clear, sticky, jelly-like semisolids or solids prepared from high molecular weight polymers in an aqueous or alcoholic base. Alcoholic gels are often drying and cooling. Non-alcoholic gels are more lubricating. Gels or jellies can be produced using a suitable gelling agent including, but not limited to, gelatin, tragacanth, a carbomer, or a cellulose derivative and may include glycerol as a humectant, an emollient, and/or a preservative. In some embodiments, gel formulations will include the same or similar ingredients as a solution or dispersion, with the addition of a gelling agent.

The gel can include a nonionic copolymer gelling agent. In one embodiment, the gelling agent is a nonionic polyoxyethylene-polyoxypropylene copolymer gel, for example, a Pluronic gel such as Pluronic F-127 (BASF Corp.), to provide a pluronic gel-based formulation. This gel is a liquid at low temperatures but rapidly sets at physiological temperatures, which confines the release of the agent to the site of application or immediately adjacent that site. Other formulations can be carboxymethylcellulose (CMC)-based formulations, hydroxy methyl cellulose (HMC)-based formulations, hydroxypropyl cellulose (HPC)-based formulations, or hydroxypropylmethylcellulose (HPMC)-based formulations, and the like.

Creams are viscous liquids or semisolid emulsions, either oil-in-water or water-in-oil. Cream bases are water-washable, and comprise an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant (a substance, such as glycerin, sorbitol, or urea, that absorbs or helps another substance retain moisture).

The emulsifier in a cream formulation is generally a nonionic, anionic, cationic, or amphoteric surfactant. Examples of emulsifiers include, but are not limited to, fatty alcohol polyoxyethylene ether (Peregal A-20), stearates such as polyoxylstearate (Softener SG), glyceryl stearate and pegylated forms of glyceryl stearate such as PEG-5 glyceryl stearate, cetyl alcohol, dithranol, or a combination thereof.

Oil-phase ingredients can include, but are not limited to, dimethicone, dimethiconol, cyclomethicone, diisopropyl adipate, cetyl alcohol, stearyl alcohol, paraffin, petrolatum, almond oil, stearic acid, or a combination thereof. In particular aspects, aqueous ingredients can include, but are not limited to, purified water, glycerol (glycerin), propylene glycol, ethyl paraben, a humectant, or a combination thereof. In some embodiments, the cream further comprises one or more film formers including but not limiting to polyglycerylmethacrylate, acrylates/Cio-Cso alkyl acrylate cross-polymers; antioxidant including but not limiting to tocopheryl acetate;

preservatives including but not limiting to phenoxyethanol, benzyl alcohol; other additives including but not limiting to dicaprylyl ether, disodium EDTA, sodium hydroxide, and lactic acid.

In one embodiment, the cream can include purified water, polyglycerylmethacrylate, propylene glycol, petrolatum, dicaprylyl ether, PEG-5 glyceryl stearate. glycerin, dimethicone, dimethiconol, cetyl alcohol, sweet almond oil, acrylates/C10-C30 alkyl acrylate cross-polymers, tocopheryl acetate, phenoxyethanol, benzyl alcohol, disodium EDT A, sodium hydroxide, lactic acid, or any combination thereof.

In another embodiment, the cream can include glycerol, light liquid paraffin, soft white paraffin, dimethicone, squalane, methyl hydroxybenzoate, dichlorobenzyl alcohol, or any combination thereof.

Ointments are semisolid preparations that include the cannabinoid base incorporated into a fatty, waxy, or synthetic base. Ointments are typically based on petrolatum or other petroleum derivatives. The specific ointment base to be used, as will be appreciated by those skilled in the art, is one that will provide for suitable cannabinoid delivery and other desired characteristics such as emolliency or the like. As with other carriers or vehicles, an ointment base is typically inert, stable, non-irritating and non-sensitizing.

Ointment bases may be generally grouped in four classes: oleaginous bases; emulsifiable bases; emulsion bases; and water-soluble bases. Oleaginous ointment bases can include, for example, vegetable oils, fats obtained from animals such as emu oil, and semisolid hydrocarbons obtained from petroleum. Emulsifiable ointment bases, also known as absorbent ointment bases, contain little or no water and can include, for example, hydroxystearin sulfate, anhydrous lanolin, and hydrophilic petrolatum.

Emulsion ointment bases are either water-in-oil (W/O) emulsions or oil-in-water O/W) emulsions, and the oil components can include, for example, cetyl alcohol, glyceryl monostearate, lanolin, and stearic acid. Water-soluble ointment bases can be prepared from polyethylene glycols of varying molecular weight.

Lotions are liquid or semiliquid preparations in which solid particles, including the active agent(s), are present in a water or alcohol base. Lotions are usually suspensions of solids, and can include a liquid oily emulsion of the oil-in-water type. Lotions are often desirable formulations because of the ease of applying a more fluid composition. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the cannabinoid stock is suspended in a suitable base. Depending on the nature of the base, pastes are divided between fatty pastes or those made from a single-phase aqueous gel. The base in a fatty paste is generally petrolatum, hydrophilic petrolatum, or the like. The pastes made from single-phase aqueous gels generally incorporate carboxymethylcellulose or the like as a base.

Foam preparations may be formulated to be delivered from a pressurized aerosol canister, via a suitable applicator, using inert propellents. Suitable excipients for the formulation of the foam base include, but are not limited to, propylene glycol, emulsifying wax, cetyl alcohol, and glyceryl stearate. Potential preservatives include methylparaben and propylparaben.

Accordingly, the cosmetic delivery system described herein may be formulated for any desired form of topical or transdermal administration. Formulations may include known antioxidants (e.g., vitamin E); buffering agents; lubricants (e.g., synthetic or natural beeswax); sunscreens (e.g., para-aminobenzoic acid); and cosmetic agents (e.g., coloring agents, fragrances, essential oils, moisturizers, or drying agents).

Auxiliary agents such as casein, gelatin, albumin, or sodium alginate may also be included in various cosmetic formulations that make up the cosmetic delivery system. Adjuvants such as fragrances and additional antimicrobial agents can be added to optimize the properties for a given use. Examples of fragrances include Ylang-Ylang oil, lavender oil, powder scent, jasmine, gardenia oil, or green tea oil. In addition, substances such as wetting or emulsifying agents, stabilizing agents, or pH buffering agents, may also be included. When a water-based carrier is used, the composition is typically near a neutral pH(+/−about 1 or 2, pH units).

Further examples of dermatological ingredients and compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.).

The cannabinoid base is admixed into a desired amount of cosmetic formulation ingredients using principles of geometric dilution until a smooth and uniform suspension is formed that is combined with other ingredients to form a gel, a jelly, a cream, an ointment, a wax, a lotion, a paste, a foam, or an aerosol. The suspension, or a gel, jelly, cream, ointment, wax, lotion, or paste can also be incorporated into a patch, such as an occlusive patch, to further improve transdermal penetration.

As a cream or lotion the cosmetic delivery system can be formulated for dispensing in a variety of ways such as squeeze dispenser, pump dispenser, roll on dispenser, tube or jar.

As a liquid the cosmetic delivery system can be dispensed as a spray, aerosol spray or foam.

Gels can be formulated for use as a lubricant. Lubricants may be water-based, silicone based, a hybrid of water/silicone, oil based and organic.

Balms and ointments can be formulation for use dispensed via a pump dispenser or tube.

Solids can be formulated as salve or stick, or suppository.

The invention provides a system to make a cosmetic delivery system for topical use for transdermal administration of a cannabinoid in a passive delivery to alleviate/manage targeted pain and general pain, soreness and inflammatory pain.

In a further embodiment of the invention, formulations of the present invention are described below in a non-limiting manner. A formulation for application to the skin, the formulation comprising: (a) a biologically active agent and (b) a lipid. The biologically active agent is selected from the group comprising Dronabinol (2), Nabiximols, Nabilone, THC, CBD, Cannabidiol, Levonantradol Ajulemic acid, (CT3), ECP002A, Natural Δ9-THC, Cannabichromenes, Cannabichromene (CBC), Cannabichromenic acid (CBCA), Cannabichromevarin (CBCV), Cannabichromevarinic acid (CBCVA), Cannabidiol Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoins, Cannabielsoic acid B (CBEA-B), Cannabielsoin (CBE), Cannabielsoin acid A (CBEA-A), Cannabigerols, Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA, Cannabinols and cannabinodiols, Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriols, 10-Ethoxy-9-hydroxy-delta-6a-tetrahydrocannabinol,8,9-Dihydroxy-delta-6a-tetrahydrocannabinol, Cannabitriol (CBT), Cannabitriolvarin (CBTV), Delta-8-tetrahydrocannabinols, Delta-8-tetrahydrocannabinol (Δ⁸-THC), Delta-8-tetrahydrocannabinolic acid (Δ⁸-THCA), Delta-9-tetrahydrocannabinols, Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-terahydrocannabivarin (THCV), Delta-9-tetrahydrocannabivarinic acid (THCVA) 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triol-THC), 3,4,5,6-Tetrahydro-7-hydroxy-alpha-alpha-2-trimethyl-9-n-propyl-2,6-methano-2H-1-benzoxocin-5-methanol, or OH-iso-HHCV.

The formulation comprises the lipid and biologically active agent in a ratio from about 5:1 to about 1:5. It is further herein acknowledged that in some embodiments of the present invention the formulation comprises the lipid and biologically active agent in a ratio from about 3:1 to about 1:3.

It is further herein acknowledged that in some embodiments of the present invention the formulation comprises a stabilizer, said stabilizer having at least one surfactant selected from the group consisting of non-ionic, anionic, cationic, and amphiphilic.

It is further herein acknowledged that in some embodiments of the present invention the formulation the non-ionic surfactant is selected from the group consisting of a polyethylene glycol derivative and a glycerol derivative.

It is further herein acknowledged that in some embodiments of the present invention the polyethylene glycol derivative is selected from the group consisting of alpha-Hydro-omega0hydroxypoly-(oxy-1,2-ethanediyl), Polyethylene glycol mono[4-(1,1,3,3-tetramethylbutyl) phenyl]ether, O-3-Amino-3-deoxy-D-glucopyranosyl-(14)-O-[2,6,diamino-2,3,6-trideoxy-D-ribo-hexopyransol-(16)]-2-deoxy-L-streptamine, alpha-hydro-omega-hydroxpoly(oxyethylene)poly(oxypropylene)poly(oxyethylene) block copolymers, Polyethylene glycol fatty alcohol ethers, Sorbitan fatty acid esters, poloxamer, and polyethylene glycol esters of fatty acids.

It is further herein acknowledged that in some embodiments of the present invention the glycerol derivative is selected from the group consisting of alpha-hydro-omega-hydroxypoly(oxy-1,2-ethanediyl) and polyalkylglyceride.

It is further herein acknowledged that in some embodiments of the present invention the anionic surfactant is selected from the group consisting of carboxylate, alkyl sulfonate, aryl sulfonate and phosphate.

It is further herein acknowledged that in some embodiments of the present invention the cationic surfactant is selected from the group consisting of alkyl pyridinium salt and tetraalkylammonium salt.

It is further herein acknowledged that in some embodiments of the present invention the amphiphilic surfactant is selected from the group consisting of alkyl betaine derivative, cocoamphodiacetate derivative, trimyristin, trilaurin, tripalmitin, tristearin, and phosphatidylglycerol.

It is further herein acknowledged that in some embodiments of the present invention the at least one lipid additive is selected from the group consisting of triglyceride, alkyl ester, cholesterol, octadecenoic acid 1,2,3-propanetriyl ester, edible oil, tetradecanoic acid 1-methylethyl ester, and methyl ester beta-Cholest-5-en-3-ol.

It is further herein acknowledged that in some embodiments of the present invention the formulation further comprises at least one additive selected from the group consisting of flavor, aroma modifier, sweetener, color, and antioxidant.

It is further herein acknowledged that in some embodiments of the present invention the lipid is in a colloidal dispersion of micelles, mixed micelles, and micellar aggregates, the lipid having a particle size of from about 10 to about 300 nm inclusive of any integer within this range. It is further herein acknowledged that in some embodiments of the present invention the lipid is in the form of a dispersion having liquid particles of size in the range of from about 50 to 300 nm.

In some embodiments of the formulation the biologically active agent is further characterized by having systemic activity, said activity being suitable for treatment of at least one condition selected from the group consisting of inflammation, irritation, dryness, and microbial infection, nausea and vomiting due to chemotherapy, appetite stimulation in HIV/AIDS, chronic pain, spasticity due to multiple sclerosis or paraplegia, depression, anxiety disorder, sleep disorder, psychosis, glaucoma, Tourette syndrome, neuropathic pain (central, peripheral, or not specified), cancer pain, diabetic peripheral neuropathy, fibromyalgia, refractory pain due to MS or other neurological conditions, rheumatoid arthritis, non-cancer pain (nociceptive and neuropathic), central musculoskeletal problems, and chemotherapy-induced pain.

The formulations comprise nano-sized lipid based high cannabinoid load capacity carrier. These formulations include an amphiphilic lipid carrier in the form of a colloidal composition which can include a micellar aggregate or mixed micelles dispersed in a continuous aqueous phase, or lipid droplets suspended in a continuous lipid phase (emulsions), and an active agent which is a cannabis derived product or cannabinoids. The formulations provide trans-dermal systemic delivery of a large amount of the cannabinoids at a controlled and prolonged manner.

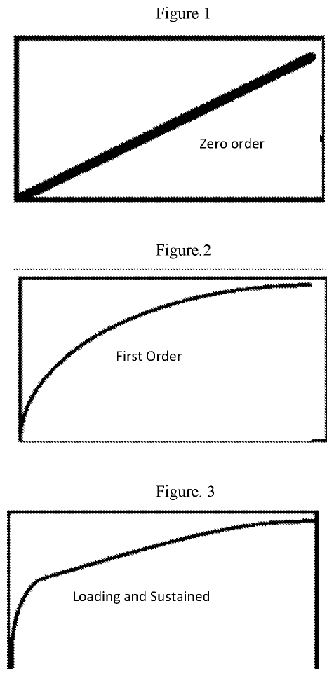

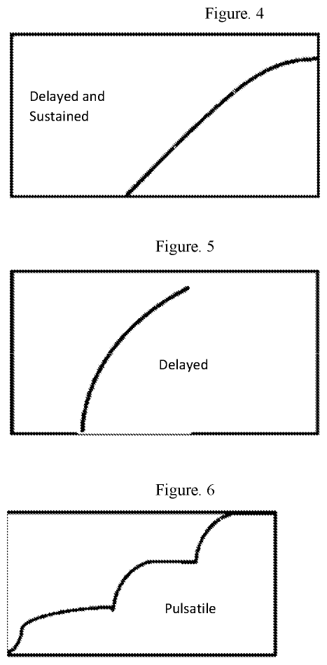

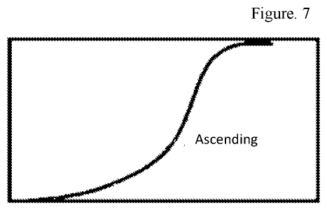

It is herein acknowledged that the present invention provides novel formulations for release of predetermined release of cannabinoids from the sub dermal lipid layer in zero order (FIG. 1), first order (FIG. 2), loading and sustained (FIG. 3), delayed and sustained (FIG. 4), delayed (FIG. 5), pulsatile (FIG. 6) and ascending (FIG. 7) manner, or any combination thereof.

It is herein acknowledged that formulations of the present invention provide trans-dermal systemic delivery of cannabinoids from the sub dermal lipid layer in a zero order (FIG. 1) release manner such that a constant amount of drug is eliminated per unit time independent of the total drug concentration in the plasma. Zero order kinetics are rare yet it is within the scope of the present invention to provide such formulations.

It is herein acknowledged that formulations of the present invention provide trans-dermal systemic delivery of cannabinoids from the sub dermal lipid layer in a first order (FIG. 2) release manner such that a constant amount of drug is eliminated per unit time dependent of the total drug concentration in the plasma,

It is herein acknowledged that formulations of the present invention provide trans-dermal systemic delivery of cannabinoids from the sub dermal lipid layer in a loading and sustained (FIG. 3) release manner after cannabinoids have been deposited in depots of the WDA to a predetermined loading concentration and then released in a sustained manner such that a constant amount of drug is eliminated per unit time dependent of the total drug concentration in the plasma.

It is herein acknowledged that formulations of the present invention provide trans-dermal systemic delivery of cannabinoids from depots in the sub dermal lipid layer in a delayed and sustained (FIG. 4) release manner such that, after a predetermined latent period of the cannabinoid formulation residing in the depots of the WDA a predetermined amount of drug is released into the patient vascular or lymphatic system, and a constant amount of drug is eliminated per unit time dependent of the total drug concentration in the plasma over a sustained period of time.

It is herein acknowledged that formulations of the present invention provide trans-dermal systemic delivery of cannabinoids from the sub dermal lipid layer depots in a delayed (FIG. 5) manner such that a constant amount of drug is released per unit time into the patient vascular or lymphatic system.

It is herein acknowledged that formulations of the present invention provide trans-dermal systemic delivery of cannabinoids from the sub dermal lipid layer in a pulsatile (FIG. 6) release manner such that several pulses of a constant amount of drug is released per unit time into the patient vascular or lymphatic system, with different pulse characteristics over time as illustrated in the graph.

It is herein acknowledged that formulations of the present invention provide trans-dermal systemic delivery of cannabinoids in an (FIG. 7) ascending manner from the sub dermal lipid layer depots of the WDA such that an increasing amount of drug is released such that a constant amount of drug is eliminated per unit time dependent of the total drug concentration in the plasma.

FIGS. 1-7 illustrate plasma concentration v. time achieved by various formulations of the present invention. Cannabinoids were measured in whole blood and/or plasma samples by conventional cannabinoid measuring techniques such as those reported in Intra-and Intersubject Whole Blood/Plasma Cannabinoid Ratios Determined by 2-Dimensional, Electron Impact GC-MS with Cryofocusing, Clin Chem. PMC 2011 Oct. 18, Clin Chem. 2009 June; 55(6): 1188-1195.

Further embodiments of the formulation of the invention comprise a transdermal patch in combination with cannabinoid formulations for carrying cannabis-based medicaments and products containing cannabis-derived products and/or cannabinoids and related compounds through the epidermis to the sub dermal lipid layer, such as the white adipose tissue (WDA).

It is herein acknowledged that embodiments of the present invention comprise a combination product, as classified by the US Food and Drug Administration. Combination products of the present invention consist of a medical device combined with formulations of cannabinoids that the device is designed to deliver.

In embodiments of the invention is a medicated cream containing cannabinoids is provided which, when applied to the skin, is able to deliver a specific dose of cannabinoids through the skin and into the bloodstream.

In embodiments of the present invention is a medicated gel containing cannabinoids is provided which, when applied to the skin, is able to deliver a specific dose of cannabinoids through the skin and into the bloodstream.

### Transdermal Patch

It is herein acknowledged that in embodiments of the present invention transdermal patch is a medicated cannabinoid dispensing adhesive patch that is placed on the skin to deliver a specific dose of cannabinoids through the skin and into the bloodstream.

It is herein acknowledged that an advantage of a transdermal drug deliver), route over other types of medication delivery such as oral, topical, intravenous, intramuscular, etc. is that the patch provides a controlled release of the cannabinoid formulations into the patient.

It is herein acknowledged that the aforementioned cannabinoid formulations are made available for topical administration via transdermal patches through a porous membrane covering a reservoir of medication or through body heat melting thin layers of cannabinoid formulation embedded in the adhesive of the transdermal patch. The main disadvantage to transdermal delivery systems stems from the fact that the skin is a very effective harrier; as a result, only medications whose molecules are small enough to penetrate the skin can be delivered by this method.

It is herein acknowledged that components to a transdermal patch of the present invention may comprise:
Liner—Protects the patch during storage. The liner is removed prior to use.
Cannabinoid based formulation—Drug solution in direct contact with release liner.
Adhesive—Serves to adhere the components of the patch together along with adhering the patch to the skin
Membrane—Controls the release of the cannabinoids based formulation from the reservoir and multi-layer patches
Backing—Protects the patch from the outer environment
Permeation Enhancer—These are permeation promoters for drugs, which increases delivery of drug, in the case of the present invention, the drug comprises Cannabinoids based formulation.
Matrix Filler—It provides bulk to matrix as well as some of fillers acts as matrix stiffening agent.

### Single Layer Drug Adhesive Patch

Embodiments of the present invention may comprise cannabinoid based formulations included in the adhesive layer of a transdermal patch. In this type of patch the adhesive layer not only serves to adhere the various layers together, along with the entire system to the skin, but is also responsible for the releasing of the drug. The adhesive layer is surrounded by a temporary liner and a backing.

### Multi-Layer Drug-in-Adhesive Patch

It is herein acknowledged that embodiments of the present invention may comprise Cannabinoids based formulation of the present invention included in a multi-layer drug-in-adhesive patch similar to the single-layer system; the multi-layer system is different, however, in that it adds another layer of drug-in-adhesive, usually separated by a membrane (but not in all cases). One of the layers is for immediate release of the drug and other layer is for control release of drug from the reservoir. This patch also has a temporary liner-layer and a permanent backing. The drug release from this depends on membrane permeability and diffusion of drug molecules.

### Reservoir Patch

It is herein acknowledged that embodiments of the present invention may comprise a cannabinoid based formulation/stock of the present invention included in a reservoir transdermal system. Unlike the single-layer and multi-layer drug-in-adhesive systems, the reservoir transdermal system has a separate drug layer. The drug layer is a liquid compartment containing a drug solution or suspension separated by the adhesive layer. The drug reservoir is totally encapsulated in a shallow compartment molded from a drug-impermeable metallic plastic laminate, with a rate-controlling membrane made of a polymer like vinyl acetate on one surface. This patch is also backed by the backing layer. In this type of system the rate of release is zero order.

### Matrix Patch

It is herein acknowledged that embodiments of the present invention may comprise cannabinoid based formulation of the present invention included in a matrix. The matrix system has a drug layer of a semisolid matrix containing a drug solution or suspension. The adhesive layer in this patch surrounds the drug layer, partially overlaying it. Also known as a monolithic device.

The descriptions of the various embodiments and/or examples of the present invention have been presented for purposes of illustration but are not intended to be exhaustive or limited to the embodiments and/or examples disclosed. Many modifications and variations will be apparent to those of ordinary skill in the art without departing from the scope and spirit of the described embodiments. The terminology used herein was chosen to best explain the principles of the embodiments, the practical application, or to enable further understanding of the embodiments disclosed herein.

The present invention will now be described in more detail with the following non-limiting Examples:

### WORKING EXAMPLES

### Example 1—Non-medicated Colloidal Composition for Evaluation of Bioadhesive Behavior on the Skin of the Upper Arm Under a Dressing

315 mg of pure phosphatidylcholine and 80 mg of polyoxyethylated sorbitan monolaurate (Tween-20) were dissolved in 2 ml of ethyl alcohol to form a solution. The solution was diluted with purified water to a final volume of 100 ml and then passed through a 0.22 micron PTFE membrane filter. The resultant colloidal carrier had a mean droplet size of about 185 nm.

The bioadhesive properties were examined according to the following method, using the radioactive Tc⁹⁹ label, which is safe and approved for human use. The lipid colloidal particles were labeled with Tc⁹⁹ by using potassium pertechnate-Tc⁹⁹, after reduction by Sn²⁺ so that substantially all radioactivity was completely associated with lipid aggregates. A water solution of Tc⁹⁹ complexed with DTPA (Diethylenetriamine pentaacetic acid), in which all radioactivity was in the aqueous phase, was used as a control. 10 ml of either the labeled colloidal composition or the control solution was administered to the upper arm of the volunteer human subject, and was then rinsed in a shower. More than 20% of the radioactive label associated with the colloidal carrier remained attached to the upper arm skin over 2.5 hours after the shower. By contrast, the radioactive label level for the control water solution dropped below 20% of its initial value after less than 20 minutes following rinse, and the remaining radioactivity detected was extremely low after this time.

Other methodologies for assessing transdermal penetration of an active are found in: Walters K A, Watkinson A C, Brain K R. In vitro skin permeation evaluation: the only realistic option. Int J Cosmet Sci. 1998; 20(5):307-316; Franz T J, Lehman P A, Raney S G. Use of excised human skin to assess the bioequivalence of topical products. Skin Pharmacol Physiol. 2009; 22(5):276-286; Yang Y, Manda P, Pavurala N, Khan M A, Krishnaiah Y S. Development and validation of in vitro-in vivo correlation (IVIVC) for estradiol transdermal drug delivery systems. J Control Release. 2015; 210:58-66; Sandby-Møller J, Poulsen T, Wulf H C; Küchler S, Strüver K, Friess W. Reconstructed skin models as emerging tools for drug absorption studies. Expert Opin Drug Metab Toxicol. 2013; 9(10):1255-1263; and Cross S E, Roberts M S. Use of in vitro human skin membranes to model and predict the effect of changing blood flow on the flux and retention of topically applied solutes. J Pharm Sci. 2008; 97(8):3442-3450. (the disclosures of each are incorporated herein by reference in their entirety).

### Example 2—CBD Colloidal Self-Emulsifying Composition

The following was mixed together: 450 mg (0.6 mmol) of purified egg lecithin; 150 mg (0.25 mmol) of CBD; 150 mg of PEG-10 laurate; and 450 mg (0.5 mmol) of triolein, and heated to 60° C. for 20 minutes until dissolution. Water was then added to this solution with gentle stirring. Immediately, a fine oil-in-water emulsion was formed. Such emulsions were observed to be stable with final oil phase concentrations of 5%-25%. The resultant emulsion can be treated by sonication, extrusion or high-pressure homogenization to standardize the size of emulsion droplets.

### Example 3—CBD Colloidal Self-Emulsifying Composition

A self-emulsifying composition containing CBD was prepared as described except 150 mg of Tyloxapol was added instead of PEG-10 laurate. After formation of the emulsion, the mixture was treated by high-pressure homogenization (6 cycles, 800 bar), producing a stable emulsion.

It is herein acknowledged that in some embodiments of the present invention the droplets, particles or micelles comprising the cannabis derived product or cannabinoids are dispersed in a continuous phase, making up to 40% by weight of the total solution.

### Example 4—Cannabinoid Stock Formation

| LIPID PHASE A | Grams |
|---|---|
| CBD (>99% purity) | 10.0 |
| Phosphatidylcholine (Phospholipid 90H) | 44.9 |
| Propylene Glycol | 44.9 |
| Vitamin E Derivative (TPCG) | 0.2 |
| | 100.0 |

Process: Heat Propylene Glycol to 55° C., add Phospholipid 90H slowly with stirring, mix until dissolved, add the CBD slowly, mixing well and add the TPCG.

| WATER PHASE B | Grams |
|---|---|
| Water | 300 |

Process: Heat the water to about 45° to about 50° C.

### Phase C

Process: Add Lipid phase A with water phase B using a high shear mixing followed by passing the mixture through a High Pressure homogenizer (e.g. Avestin™ EmulsiFlex-C-50) at pressures ranging from about 5,000 to about 20,000 psi. This process is repeated for several cycles until the emulsion to achieve a smooth and uniform emulsion system.

This emulsion is used as a base material to provide a fast and effective CBD and/or THC delivery system.

The emulsion was added in desired ratios to a cosmetic format that is a cream, balm, lotion, gel, ointment, liquid, oil or solid forming the product for topical use. Example: Topical cream/gel for pain relief, or for vaginal lubricant applications. Some products will be formulated for the specific applications as creams or spray. It is herein acknowledged that in some embodiments of the present invention the droplets, the non-lipid portion of the solution is a hydrogel.

### Example 5—Lubricant Cosmetic Format Formulation

| | (%) |
|---|---|
| Water | 75.53 |
| Natrosol 250HHR | 0.8 |
| Vanzan NF | 0.15 |
| Aloe Vera Gel | 2.0 |
| Zemea (Propanediol) | 2.0 |
| Hemp Extract | 0.1 |
| Sodium Hyaluronate 1% | 1.0 |
| Quinoa Seed Extract GL | 0.1 |
| Cannabinoid Stock - CBD 10% solution | 16.0 |
| Linseed Extract GL | 0.1 |
| Green Tea Extract GL | 0.1 |
| Shitake Mushroom Extract | 0.1 |
| Oat Kernel Extract GL | 0.1 |
| Citric Acid 20% solution | 0.15 |
| Geogard Ultra | 1.0 |
| Potassium Sorbate | 0.3 |
| NaOH 20% solution | 0.37 |
| Sodium Benzoate | 0.1 |
| (uric acid to pH to 5.0-5.5). | 100.00 |

### Example 6—Pain Relief Gel 150 mg CBD

| | (%) |
|---|---|
| Water | 67.6 |
| Lecigel (Sodium acrylates copolymer and lecithin) | 2.0 |
| Glycerin | 1.5 |
| Allantoin | 0.15 |
| Sodium Hyaluronate | 0.5 |
| CBD Stock lipid 10% | 16.0 |
| Camphor | 0.5 |
| Allantoin | 0.2 |
| Menthol | 10.0 |
| Vitamin E Acetate (Tocopherol acetate) | 0.25 |
| Arnica Extract GL | 0.10 |
| Boswellia Extract GL | 0.10 |
| Aloe Veral Gel 10x (Ale Barbadensis Leaf Juice) | 0.10 |
| Euxyl PE 9010 | 1.0 |
| | 100.0% |

### Example 7—Cream 200 mg CBD

| | |
|---|---|
| CBD Stock lipid 10% | 16.0 |
| Water | 67.6 |

One or more of Cetearyl Ethylhexanoate, Helianthus Annuus Seed Oil, Distarch Phosphate, Cetearyl Alcohol, Dimethicone, Cetearyl Glucoside, Parfum, Phenoxyethanol, Potassium Olivoyl Hydrolyzed Oat Protein, Glyceryl Stearate, Allantoin, Acrylates/C10-30 Alkyl Acrylate Crosspolymer, Cannabidiol, Glyceryl Oleate, Ethylhexylglycerin, Benzyl Alcohol, Potassium Sorbate, Sodium Benzoate, Alpha Isomethyl Ionone, and Benzyl Salicylate 17%.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

It will be appreciated that the above descriptions are intended only to serve as examples, and that many other embodiments are possible within the spirit and the scope of the present invention.

1. A cannabinoid stock comprising a stabilized cannabinoid/lipid particle emulsion, wherein the lipid and the cannabinoid are present in a ratio from about 5:1 to about 1:5.
